(19) United States
(12) Patent Application Publication
(10) Pub. No.: US 2023/0110978 A1
(43) Pub. Date: Apr. 13, 2023

(54) ENHANCED MULTICORE FIBER ENDOSCOPES

(71) Applicant: ZSQUARE LTD., Petah Tikva (IL)

(72) Inventors: Zeev ZALEVSKY, Rosh Ha Ayin (IL); Asaf SHAHMOON, Petah-Tikva (IL); Amihai MEIRI, Ramat Hasharon (IL)

(73) Assignee: ZSQUARE LTD., Petah Tikva (IL)

(21) Appl. No.: 18/080,844

(22) Filed: Dec. 14, 2022

Related U.S. Application Data
(62) Division of application No. 16/221,593, filed on Dec. 17, 2018.

(51) Int. Cl. A61B 1/06; A61B 1/07; A61B 1/04; A61B 1/00

(52) U.S. Cl. CPC A61B 1/063; A61B 1/07; A61B 1/0669; A61B 1/042; A61B 1/000094; A61B 1/00167

(57) ABSTRACT

endoscope includes an illumination source for generating a coherent laser illumination beam; an optical sensor; a multicore fiber comprising: at least one core for transferring the illumination beam from the illumination source through said at least one core to a distal end of the fiber, for illumination of a surface to be inspected; and a plurality of cores for transferring light reflected off the surface to the optical sensor; a temporal modulation sequencer for separating a specular image of the illumination beam from an image of the surface; and a processor, for processing sensed data from the optical sensor to generate the image of the surface.

### CROSS-REFERENCE

The present application is a divisional application of U.S. patent application Ser. No. 16/221,593, filed Dec. 17, 2018, incorporated herein in its entirety.

### FIELD OF THE INVENTION

The present invention relates to endoscopy and more particularly to multicore fiber endoscopes.

### BACKGROUND OF THE INVENTION

Endoscopes are widely used in medical diagnostics and in other medical procedures, aimed at providing the medical expert, or medical team, with a clear image of examined internal hollow organ or cavity inside the patient's body.

There are various known kinds of endoscopes. Typically, an endoscope is made up of a flexible tube that includes an optical guide for transmitting light from an illumination source to the distal end of tube, which is inserted into the patient's body, so as to illuminate the inspected surface inside the body, and an optical guide for transmitting reflected light off the illuminated surface to an eyepiece or to an optical sensor that is a part of an optical system for processing the reflected light and displaying an image of the illuminated surface. Many endoscopes employ optical fibers as their optical guides for illumination as well as for transmission of the light reflected of the inspected surface inside the body.

Multicore fiber endoscopes are known, which include a bundle of optical fibers, which are used for transferring light reflected off the inspected surface to the eyepiece or to the display device.

### SUMMARY OF THE INVENTION

There is thus provided, according to some embodiments of the present invention, an endoscope. The endoscope may include an illumination source for generating a coherent laser illumination beam. The endoscope may also include an optical sensor. The endoscope may also include a multicore fiber comprising: at least one core for transferring the illumination beam from the illumination source through said at least one core to a distal end of the fiber, for illumination a surface to be inspected; and a plurality of cores for transferring light reflected off the surface to the optical sensor.

According to some embodiments, the temporal modulation sequencer comprises two Faraday rotators, each having a rotation axis, wherein the rotation axes are substantially parallel but offset.

According to some embodiments, the two Faraday rotators are configured to produce a lock-in amplification effect.

According to some embodiments, there is provided an endoscope comprising a multicore fiber with a plurality of cores, the cores having at least two different cross section shapes or orientations, wherein said at least two different cross section shapes or orientations are arranged in a mixed arrangement.

According to some embodiments, said at least two different cross section shapes or orientations are arranged in interlaced arrays.

According to some embodiments, an elongated axis of the cross section of cores of one of said at least two different cross section shapes or orientations is orthogonal to an elongated axis of another of said at least two different cross section shapes or orientations.

According to some embodiments, there is provided an endoscope comprising: a multicore fiber comprising: an illumination region that includes at least one :hollow core for transferring an illumination beam from an illumination source through said at least one core to a distal end of the fiber, for illumination of a surface to he inspected, and a plurality of cores surrounding each of said at least one hollow core for trapping the illumination beam in said at least one hollow core; and an imaging region that includes a plurality of cores for transferring light reflected off the surface to the optical sensor.

According to some embodiments, the plurality of cores surrounding each of said at least one hollow core are arranged in a plurality of rings around that hollow core.

According to some embodiments, the plurality of rings comprises at least three rings.

According to some embodiments, the plurality of cores surrounding each of said at east one hollow core comprise photon crystal fibers (PCF).

There is also provided an endoscope that may include an optical sensor; a multicore fiber comprising: a plurality of cores for transferring light reflected off an inspected surface to the optical sensor; a lens for performing Fourier transform on the light reflected off the surface placed at a predetermined distance from a distal tip of the multicore fiber; a lens for performing inverse Fourier transform on the light reflected off the surface after it emerges from a proximal tip of the multicore fiber, placed at a predetermined distance from a proximal tip of the multicore fiber; and a processor for processing sensed data from the optical sensor to generate the image of the surface.

According to some embodiments, the endoscope may further include a laser source for generating a reference illuminating beam to realize a digital Fourier holographic recording so as to construct a phase and of an image of the inspected surface by digital decoding.

According to some embodiments, the endoscope may also include an optical element that includes a plurality of PCF cores, placed on the optical path of the :light reflected off the inspected surface into the multicore fiber.

According to some embodiments, there is provided an endoscope comprising: a multicore fiber comprising: an illumination region that includes at least one imaging core for transferring an illumination beam from an illumination source through said at least one core to a distal end of the fiber, for illumination a surface to be inspected, an imaging region that includes a plurality of cores for transferring light reflected off the surface to the optical sensor; and a dye container for containing a fluorescent dye configured to supply the dye into said one imaging core, so as to allow irradiating the dye to generate continuous white light illumination spectrum.

According to some embodiments, the dye is a solution mixture.

According to some embodiments, there is provided an endoscope that may include a multicore fiber comprising: an imaging region that includes a plurality of imaging cores for transferring light reflected off a surface to an optical sensor; a plurality of surrounding cores about each of said plurality of imaging cores, wherein a diameter of each of the surrounding cores is smaller than the wavelength of reflected light anticipated to traverse through the imaging cores.

According to some embodiments, the surrounding cores are hollow air-filled cores.

According to some embodiments, there is provided a method for increased radiation safety in endoscopy that uses laser light, the method may include embedding amplification material into imaging cores of a multicore fiber of an endoscope; and pumping the imaging cores by laser light of predetermined wavelength configured to amplify light reflected off an inspected surface traversing through the imaging cores.

According to some embodiments, there is provided a method for optical zooming in endoscopy, the method comprising: using a laser source, generating an illumination beam for illuminating an inspected surface, the beam having a spherical wavefront of a predefined curvature relating to a design of a lens located at a distal tip of an endoscope multicore fiber; and changing the curvature of the spherical wavelength with respect to the design of the lens, so as to optically zoom in or zoom out on an image of the inspected surface.

According to some embodiments, there is also provided a method for optical zooming when using an endoscope for inspecting an inspected surface, the endoscope having a first Fourier lens having a focal length at a distal tip of a multicore fiber. The method may include providing a second Fourier lens having a tunable focal length at a proximal tip of the multicore fiber placed between the proximal tip and an optical detector; and tuning the tunable focal length of the second Fourier lens; so as to optically zoom in or zoom out on an image of the inspected surface.

According to some embodiments, there is also provided a method for improving resolution of an image obtained using an endoscope with a multicore fiber, the method comprising: illuminating an inspected surface by a plurality of different wavelengths of red, a plurality of different wavelengths of green and a plurality of different wavelengths of blue; collecting by an optical detector image data of light reflected off the inspected surface by the plurality of different wavelengths of red, the plurality of different wavelengths of green and the plurality of different wavelengths of blue, and constructing an image from the collected image data.

According to some embodiments, there is further provided a method for optical zooming and improving image resolution in endoscopy, the method comprising: providing a bifocal lens on an optical path of light reflected off an inspected surface and traversed through a multicore fiber that includes a plurality of imaging cores; using each of the focal lengths of the bifocal lens for calibration; based on the calibration separating two super imposed images, a regular image and a zoomed in image, obtained by the endoscope on an optical detector; digitally zooming out on the zoomed in image; and adding image data from the zoomed in image after it was zoomed out to the regular image to obtain a higher resolution regular image.

There is also provided, according to some embodiments of the invention, an endoscope that, includes a multicore fiber comprising: a plurality of imaging cores for transferring light reflected off a surface to an optical sensor, each core coated by a metallic coating.

According to some embodiments, a pitch of the cores is smaller than 2 microns.

According to some embodiments, the pitch is about 1 micron

According to some embodiments, there is also provided an endoscope that includes a multicore fiber with a distal end and a proximal end, having one or a plurality of illumination cores for transmitting light from the proximal end and out of the distal end and illuminating an internal surface of a patient to be viewed and with one or a plurality of imaging cores to transfer light reflected off the surface into the imaging cores from the distal end to the proximal end. The endoscope may also include a shielding sleeve in which the multicore fiber is inserted and a mechanism for advancing and retracting the multicore fiber within the shielding sleeve, so as to allow extracting a distal tip of the multicore fiber to contact the surface and collect a sample for analysis and retract the distal tip into the shielding sleeve.

According to some embodiments, the mechanism includes a plunger.

According to some embodiments, the endoscope includes an optical sensor or receiving the reflected light.

According to some embodiments, the optical sensor is a Raman spectrometer, for performing analysis of the sample.

### BRIEF DESCRIPTION OF THE DRAWINGS

In order the present invention to be better understood and its practical applications appreciated, the following figures are provided and referenced hereafter. It should be noted that the figures are given as examples only and in no way limit the scope of the invention. Like components are denoted by like reference numerals.

FIG. 1 is a schematic arrangement of polarization modulation which may be incorporated in an endoscope and used in some embodiments of the present invention, to eliminate or at least greatly reduce the specular image of the illumination source on the inspected surface.

FIG. 2 shows an endoscope with Faraday rotators, aimed at eliminating specular image, according to some embodiments of the present invention.

FIG. 3 shows a cross section of a multicore fiber endoscope, according to some embodiments of the present invention, with a plurality of cores of different shape and/or orientation.

FIG. 4A shows a monolithic imaging and illumination multicore fiber of an endoscope, according to some embodiments of the present invention.

FIG. 4B shows a cross section of a multicore fiber endoscope, with a plurality of metal coated cores, according to some embodiments of the invention.

FIG. 5A shows a multicore fiber for an endoscope, according to some embodiments of the invention, for obtaining high-resolution imaging.

FIG. 5B shows a multicore fiber for an endoscope, according to some embodiments of the invention, for obtaining high-resolution imaging, using optical element comprising four photon crystal fibers (like in a kaleidoscope).

FIG. 6 shows a cross section of a rectangular multicore fiber of an endoscope, with white light illumination cores, according to some embodiments of the present invention.

FIG. 7 shows cross section of several imaging cores with peripheral cores for inhibiting cross talk between the imaging cores, according to some embodiments of the invention.

FIG. 8 illustrates a method for increased radiation safety in endoscopy using laser light, according to some embodiments of the present invention.

FIG. 9 shows a method for optical zooming in endoscopy, according to some embodiments of the invention.

FIG. 10 shows a method for optical zooming in endoscopy, according to some other embodiments of the invention.

FIG. 11 shows a multicore fiber of an endoscope for providing two optical zoomed. images, according to some other embodiments of the invention.

FIG. 12A shows a multicore fiber endoscope with sampling capability, according to some embodiments of the invention, in a viewing mode.

12B shows a multicore fiber endoscope sampling capability, according to some embodiments of the invention, in a sampling mode.

FIG. 13 illustrates a method for improving resolution of an image obtained in endoscopy, according to some embodiments of the invention.

FIG. 14 shows a method for optical zooming and improving image resolution in endoscopy, according to some embodiments of the invention.

### DETAILED DESCRIPTION OF THE INVENTION

Iii the following detailed description, numerous specific details are set forth in order to provide a thorough understanding of the methods and systems. However, it will be understood by those skilled in the art that the present methods and systems may be practiced without these specific details. In other instances, well-known methods, procedures, and components have not been described in detail so as not to obscure the present methods and systems.

Although the examples disclosed and discussed herein are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method examples described herein are not constrained to a particular order or sequence. Additionally, some of the described method examples or elements thereof can occur or be performed at the same point in time.

Unless specifically stated otherwise, as apparent from the following discussions, it is appreciated that throughout the specification, discussions utilizing terms such as “adding”, “associating” “selecting,” “evaluating,” “processing,” “computing,” “circulating,” “determining,” “designating,” “allocating” or the like, refer to the actions and/or processes of a computer, computer processor or computing system, or similar electronic computing device, that manipulate, execute and/or transform data represented as physical, such as electronic, quantities within the computing system's registers and/or memories into other data similarly represented as physical quantities within the computing system's memories, registers or other such information storage, transmission or display devices.

Some embodiments of the present invention relate to improvements in endoscopes. Some embodiments of the present invention specifically relate to improvements in multicore fiber endoscopes.

The terms “distal” and “proximal” as used in this application refer to the ends of the endoscope. The end and associated parts of the endoscope which are far from the endoscope's interface (detector or eye) and close to the imaged tissue and to its surroundings are termed the distal end, while the end and associated parts of the endoscope which are close to the endoscope's interface and are remote from the imaged tissue, being typically outside the body, are termed the proximal end. The term “reflected” as used in this application refers to a change in a direction of an illumination wavefront which impacts one or more imaged object or tissue. The term “reflection” is understood broadly as any radiation gathered by the fiber, irrespective of the source of the illumination which is reflected by the object(s) and/or tissue(s).

The term “near field imaging” as used in this application refers to the formation of an image (of imaged objects, tissues and/or their surroundings) at the distal end of the endoscope fiber, typically at the fiber's tip. The imaged is then typically transferred through the fiber to the detector, possibly through proximal optical elements. The term “near field imaging” may relate to different types of optical systems, including direct imaging without any optical elements between the imaged object or tissue and the fiber tip as well as to imaging through optical elements) such as lenses.

The term “far field imaging as used in this application refers to the formation of a Fourier transform of imaged objects, tissues and/or their surroundings at the distal end of the endoscope fiber (e.g., the distal end of the endoscope fiber is at the aperture or pupil plane of the optical system), typically at the fiber's tip. The image of the imaged objects, tissues and/or their surroundings may be formed at the proximal end of the endoscope fiber, typically at the fiber's proximal tip or directly on the detector, possibly through proximal optical elements. The term “far field imaging” may relate to different types of optical systems. In one example, far field imaging” may be direct in the sense that no optical elements are used between the imaged object or tissue and the distal fiber tip, which delivers radiation entering the fiber along the fiber to the detector at the proximal end of the fiber. In another example, “far field imaging” may be carried out with optical elements positioned between the imaged object or tissue and the distal fiber tip, with the distal fiber tip being at least approximately at the Fourier plane (also termed aperture plane and pupil plane in different contexts) of the optical elements.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

Some embodiments of the present invention may involve endoscopes such as described, for example, in U.S. Pat. No. 9,661,986 (Shahmoon et al.), and/or U.S. Patent Application Publication No. 2017/0100024 (Shahmoon et al.), all incorporated herein by reference.

Embodiments of the present invention relate to endoscopes, multicore endoscope fibers and configuration and operation methods. Multicore fibers, according to some embodiments, may have a large number (e.g., hundreds or thousands) of cores, and may incorporate working channel(s) and/or additional fibers. The fiber used may be provided in different optical configurations to capture images of tissue and objects at the distal tip of the endoscope, and to enhance a wide range of optical characteristics of the obtained images, such as resolution, field of view, depth of field, wavelength ranges, etc. Near-field imaging as well as far-field imaging may be implemented in the endoscopes, according to some embodiments of the invention, and the respective optical features may be utilized to optimize imaging. Optical elements may be used at the distal tip, or the distal tip may lack any lenses. Diagnostics and optical treatment feedback loops may be implemented, and illumination may be adapted to yield full color images, depth estimation, enhanced field of view and/or depth of field and additional diagnostic data.

Various embodiments of multicore endoscope fibers may be used. An endoscope, according to some embodiments of the present invention, may implement far field imaging, i.e., have the image formed at the proximal end of the endoscope fiber, while another endoscope, according to some embodiments, may implement near field imaging, i.e., have the image formed at the distal end of the endoscope. Both far field and near field implementations, may have distal optical elements between the imaged objects or tissues and the distal tip of the endoscope, or may operate without such distal optical elements. Each of the four combinations (far field with or without distal optical elements and near field with or without distal optical elements) has different features, advantages and disadvantages, and may be selected according to specific implementation scenarios. Alternation of the combination may be carried out between applications or in real time, to combine advantages of different configuration types. It is further noted that, endoscopes may be designed to have several combinations, e.g., a part of the fiber face (or certain fiber modules) having distal optics for imaging far objects and another part of the fiber face (or other fiber modules) lacking distal optics for microscopic imaging.

Endoscopes, according to some embodiments, may lack any optical elements at the distal tip. Such lens-less embodiments may implement either far-field or near-field imaging, and may utilize structural features to enhance optical resolution, apply super-resolution methods and retrieve wavefront information while reducing crosstalk between the cores.

Endoscopes, according to some embodiments of the invention, may have full tip cross sections or have working channel(s) within the imaging fiber characterized by different configurations and uses, integrating additional fibers etc., in which case the cores and optical elements may be configured to overcome reduction of the field of view due to the incorporation of the working channel.

Various configurations of large number of cores in the endoscope fiber, according to some embodiments of the invention, may provide solutions to various issues such as reducing crosstalk between the fibers, overcoming material losses, achieving enhanced resolution by different methods, providing required mechanical characteristics and optimizing the imaging performances of the endoscope fibers. Endoscopes, according to some embodiments of the invention, may serve for different purposes, e.g., may be designed as a laparoscope or an ureteroscope, etc.

A micro endoscope, according to some embodiments of the invention, may be constructed from a large number of cores (e.g., one hundred cores or more, hundreds of cores, thousands of cores, in certain embodiments tens or hundreds of thousand cores per fiber or fiber module, reaching over a million cores in certain fiber endoscopes), where each core may be responsible for transferring a single or a large number of spatial degrees of freedom out of which at the output, proximal end (the end which is external to the patient body), a high resolution color image may be constructed. Multi-core fiber, according to some embodiments of the present invention, may exhibit a high degree of flexibility in its optical design, which may be utilized and adapted for specific applications, for example for ureteroscopes with a large working channel and a small external diameter or for laparoscopes with a very high resolution obtained at a small external diameter.

An endoscope according to some embodiments of the invention may be configured to carry out far-field imaging, near-field imaging or a combination of far-field imaging and near-field imaging. Irrespective of the imaging mode, such an endoscope may be configured to have one or more optical elements at a distal tip of the fiber or have no optical elements between the distal tip and imaged tissue(s) or object(s). Endoscopes, according to some embodiments of the invention, may include removable or reconfigurable optical elements at the distal tip and/or optical elements affecting only parts of the surface of the distal tip (e.g., sub-groups) of the cores).

According to some embodiments, endoscopes may include a plurality of fibers, grouped together, each having at least one hundred cores distributed at a fill factor smaller than ¼, or even smaller than 1/9, at least one photonic illumination fiber, and at least one optical element at a distal tip of the fibers, which may be configured to enhance a field of view and/or a depth of field of the endoscope beyond a region facing a tip of the fibers and congruent thereto. Such an endoscope may be further configured to implement three-dimensional sensing by handling the cores group-wise with respect to radiation delivered therethrough. An endoscope according to some embodiments of the invention may be further configured to perform super-resolved imaging by micro scanning over a pitch distance between the cores. Such an endoscope may be configured to comprise a LED (light emitting diode) light source located at the distal tip as an illumination source.

In far-field imaging, an image (indicating any kind of electromagnetic signal reflected from a tissue or an object) may be delivered through an endoscope distal tip and fiber to yield an image on a detector. The distal tip may constitute a Fourier plane (also termed aperture plane or pupil plane) at which a Fourier transform of the image enters the fiber. It is noted that the Fourier plane may be located anywhere along the fiber as well as distally or proximally to the fiber, in different embodiments of the invention, and may be optically transformed to an image on a detector at the proximal end of the fiber. Alternatively, or complementary, a Fourier image or derivatives thereof may be measured at the detector and/or manipulated to enhance imaging parameters such as resolution, field of view and depth of focus, as non-limiting examples. Optical elements may be introduced distally or proximally to the fiber to modify or manipulate the radiation entering the distal tip and the radiation falling on the detector, respectively.

In near-field imaging, an image may be captured at the distal fiber tip. The image may then be delivered, possibly through optical elements, to the detector through the fiber. It is noted that the image may be formed within the fiber and not necessarily at the distal tip. The image may be delivered via the fiber and may be measured at the detector, and/or manipulated to enhance imaging parameters such as resolution, field of view and depth of focus, as non-limiting examples. Optical elements may be introduced distally or proximally to the fiber to modify or manipulate the radiation entering the distal tip and the radiation falling on the detector, respectively.

Optical element(s) may be attached to the distal tip or may be spaced from the distal tip (e.g., held by spacers at a distance therefrom). Each optical element may be in optical communication with a respective core or a respective group of cores. Proximally, the illumination is delivered through the fiber and reflected illumination (e.g., in far-field or in near-field) may be directed via the cores to a detector, e.g., via a beam splitter. Proximal optical elements may be set and used to manipulate the illumination and the reflected illumination. One or more processor(s) may be configured to control the illumination and/or process the detected illumination, as well as control illumination and image beams in case there are controllable elements in the optical path.

Some embodiments of the invention may have no optical elements) (also termed herein as “lens-less” configurations) at the distal end of the fiber, so that the distal tip of the fiber may be used directly to deliver and receive illumination to and from the imaged tissue. Illumination may be delivered by the fiber proximally, e.g., via an optical element such as a lens, and reflected illumination may be directed to the detector via another optical element, e.g., a lens. One or more processor(s) may be provided, configured to control the illumination and/or process the detected illumination, as well as to control illumination and image beams in case there are controllable elements in the optical path. In some embodiments, lens-less configurations may be configured to generate image at “contact mode”, i.e., with close proximity of the distal tip of the fiber to the examined tissue, to yield microscopic resolution determined by the sizes of the cores.

In some embodiments, proximal optical elements may be used to adjust the plane and depth of focus of captured images in far-field imaging configurations, especially in lens-less configurations.

An endoscope fiber, according to some embodiments of the invention, may include a large number of cores. Such fiber may include central or eccentric optical cores and/or may have hollow, central or eccentric region(s) that may be used for treatment such as energy delivery, suction, illumination, drug delivery, etc. Illumination means may be integrated within the multicore fibers. Selection of near-field or far-field configurations, as well as selection if and which optical elements are to be placed distally to the tip, may be carried out under consideration of the tradeoffs between the different applications. For example, considerations concerning production, use, optical characteristics and algorithmic parameters may be balanced differently at different embodiments to optimize the endoscope to a wide range of performance and device requirements.

The cross section of the fiber may be round, elliptic, square, rectangular, or of other shape (e.g., polygon) etc. A hollow endoscope, according to some embodiments of the invention, may have a void within the fiber for different purposes (e.g., as a working channel for inserting a tool or carrying out suction, for incorporating additional fibers etc.).

Illumination of an endoscope, according to some embodiments, may comprise coherent light or incoherent light, any spectral pattern (broad or narrow wavelength ranges, continuous or discrete ranged), polarized (in various patterns) or non-polarized light and different ranges in the visual or infrared ranges. Material differences between cores, interspaces and outer cladding may comprise different materials, using air cores or air interspaces, and doping any of the fiber regions to influence their refractive indices, as explained in more details below. It is noted that any of the embodiments presented below may be used in any of the other embodiments described herein, as long as they are compatible. Particularly, computational methods optical methods and fiber design considerations described in the context of any embodiment may be applied to other embodiments as well.

Multi-core fibers may be made using fiber modules or units. Each fiber module is itself a multicore fiber, possibly configured to have uniform dimensions. Such embodiments are referred to as bundled fibers and may bundle any number of fiber modules in any configuration (e.g., 2×2 modules, 3×3 modules etc.). A fiber module may have any form, such as square, rectangle, circle, ellipse, and may be packed into fibers having a wide range of forms and configurations. Introducing fiber modules with an intermediate dimension between cores or core groups and whole fiber (each module may have, e.g., tens, hundreds or thousands of cores) enables simpler production and higher flexibility on forming the fiber from the fiber modules.

According to some embodiments of the present invention, an endoscope, e.g., a multicore fiber endoscope, is provided, which is aimed at eliminating or at least greatly reducing specular image associated with the illumination spot caused by the illuminating source.

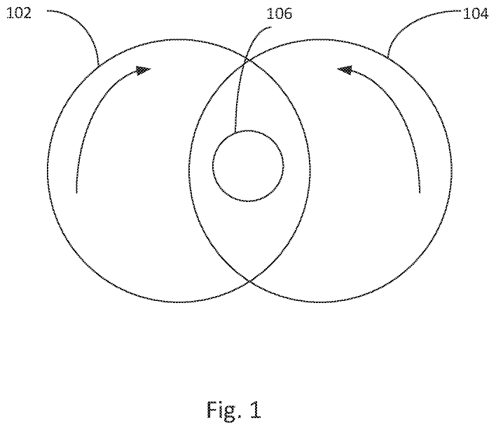

FIG. 1 illustrates a schematic arrangement of polarization modulation which may be incorporated in an endoscope and used in some embodiments of the present invention, to eliminate or at least greatly reduce the specular image of the illumination source on the inspected surface.

In the example shown in FIG. 1, two Faraday rotators 102 and 104 are provided. A Faraday rotator is a polarization rotator, based on the Faraday effect, that is able to rotate the plane of linearly polarized light. The plane of linearly polarized light may he rotated when a magnetic field is applied parallel to the propagation direction of the light.

According to some embodiments of the present invention, a temporal modulation polarization state is generated to be used for the lock-in amplification process. One possible way of generating temporal polarization modulation may be obtained, for example, by using two Faraday rotators, whose rotation axes are substantially parallel but offset. By rotating the Faraday rotators at different, known, angular velocities (e.g., different frequencies) a temporal polarization modulation sequence may be obtained on a linearly polarized laser beam, e.g., the illumination light generated by the illumination source of an endoscope.

Given that the specular image of the illumination spot is partially polarized, whereas non-specular image of the illuminated surface is not, it may be possible to use the two Faraday rotators as a lock-in amplification effect to better separate, e.g., eliminate or greatly reduce, the specular image 106 from the non-specular image of the illuminated surface, by manipulating the Faraday rotators, for example, as described hereinabove. This is because the matched encoding-decoding sequence will be applied mainly on the specular image and not on the non-specular one and thus the might better be separated from each other. In lock-in amplification effect the signal may be encoded and decoded later on, allowing the signal to be amplified differently than the noise for which there is no match between encoding-decoding sequences.

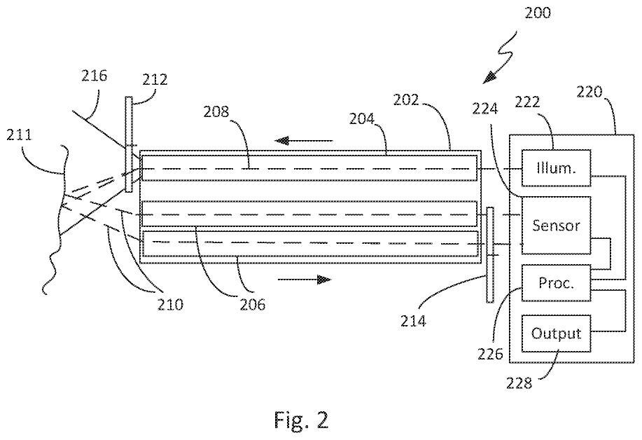

FIG. 2 shows an endoscope with Faraday rotators, aimed at eliminating specular image, according to some embodiments of the present invention.

Endoscope 200, e.g., multicore fiber endoscope, is shown, having an elongated multicore fiber 202, and a controller 220. Controller 220 may include an illumination source 222, for generating an illumination beam, e.g., a laser beam 208, to be transmitted through one or more designated illumination cores, e.g., core 204 along multicore fiber 202 and to illuminate an inspected surface 211 located in front of the distal tip of the endoscope fiber, an optical sensor 224 (e.g., an optical detector), for sensing light 210 reflected off the inspected surface 211 and transmitted to the optical sensor 224 via a plurality of cores, e.g., cores 206, a processor 226, for controlling and operating the illumination source 222, process sensed data from optical sensor 224, and generate image data for outputting via output device 228. Output device 228, may be, in some embodiments, a display device, e.g., a computer screen, for displaying an image generated by the processor, or an optical eyepiece for human eye inspection.

In some embodiments, illumination source 222 may comprise one or more laser source(s) (possibly narrowband sources) and at least one beam shaping element at the distal end of multicore fiber 204 which is configured to generate an optimized beam profile to improve illumination. For example, a beam profile may comprise a uniform illumination distribution in space or a rectangular uniform profile (e.g., top hat illumination distribution), which may be advantageous with respect to Gaussian illumination distribution with respect to various parameters of the resulting images. The coherence of the laser source may be used to shape illumination beam efficiently by the beam shaping element. In some embodiments, at least one beam shaping element may be set at the proximal end of multicore fiber 204.

In some embodiments, illumination source 222 may comprise one or more laser treatment source which are configured to apply a specified treatment by endoscope 200, e.g., to a tissue. For example, a laser treatment may be applied to kidney stones using endoscope 200 designed as an ureteroscope.

The design the endoscope, as shown in FIG. 2 (without the Faraday rotators—hereinafter—“the basic design”), according to some embodiments of the present invention, and a design of a typical endoscope, as known in the art, is applicable to any of the improvements discussed herein. The basic design of the endoscope may incorporate each of the improvements discussed herein, either separately or in various combinations. So, when describing parts of an endoscope (e.g., only the fiber, and/or other parts of an endoscope), it should be understood that these described parts may be applied to the basic design of the endoscope, as well as to other endoscopes.

According to some embodiments of the present invention, a temporal modulation sequencer is provided, for example, in the form of two Faraday rotators 212 and 214, which are placed in the optical path of the illumination beam and operable by controller 220. As shown in this figure, the Faraday rotators may be spaced apart, e.g. one Faraday rotator 212 may be placed at the distal end of the endoscope 100, at the exit of the illumination beam 208 from the illumination optical guide (optical fiber 204) and the other Faraday rotator may be placed at the proximal end of the endoscope, where the reflected beam of light 210 emerges from the optical fibers 206 in front of the optical sensor 224.

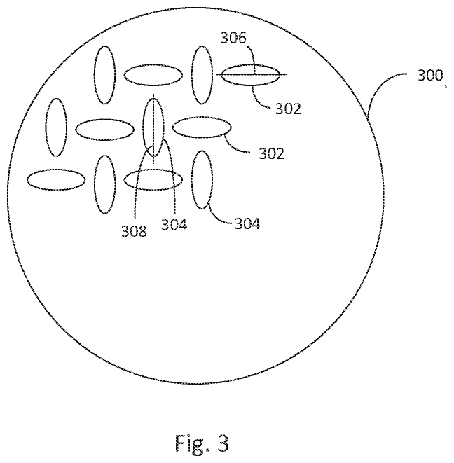

FIG. 3 illustrates a cross section of a multicore fiber endoscope, according to some embodiments of the present invention, having a multicore fiber 300 with a plurality of cores of different shape and/or orientation—302 and 304 in this example. In the arrangement depicted in this figure, cores 302 have an elongated axis 306, and cores 304 have an elongated axis 308, which is substantially orthogonal to elongated axis 306. In some embodiments of the present invention, typical core diameter may range between 0.8 to 2 microns, e.g., about 1 micron, etc. The core arrangement is illustrated only partially occupy the multicore fiber, but, in other embodiments the cores 302 and 304 either may fill the entire cross section of the multicore fiber or may occupy one or more sub-zones within the cross section. The cores are arranged in a mixed arrangement, e.g., cores 302 are arranged in one array while cores 304 are arranged in a second array which is shifted with respect to the first array. Such arrangement of cores (or photonic crystal fibers—PCFs, which for sake of brevity are also referred to herein as “cores”) may facilitate enhanced resolution and greater energetic throughput. By providing a mixed arrangement (e.g., interlaced array arrangement) of two structures (e.g., PCF cores or other cores) having different shapes denser resolution transmission and smaller cross talk between cores may be achieved. Another advantage of the proposed arrangement is that blue wavelength exhibits more cross talk to adjacent cores than red, such that, by applying such an interlaced arrangement, dispersion may be enhanced to cancel diffraction and have the same cross talk for all wavelengths which will provide better uniformity for the captured image. Another added value is that the fill factor is much larger (more cores may be confined to a fiber of a given cross section), and thus the cross-section area of the fiber is better utilized for imaging purposes.

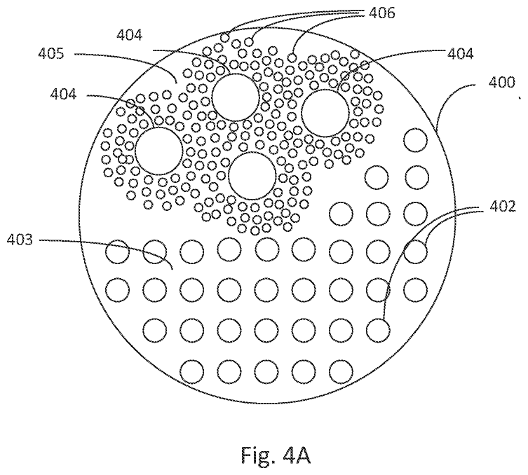

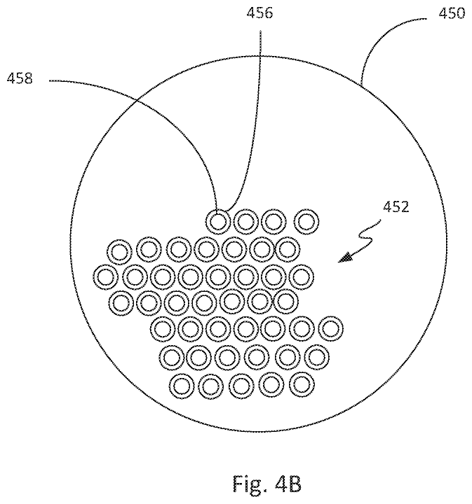

FIG. 4A illustrates a monolithic imaging and illumination multicore fiber 400 of an endoscope, according to some embodiments of the present invention.

The monolithic fiber 400 of the endoscope may comprise at least one (or more) imaging multi core region 403 with a plurality of imaging cores 402 and at least one (or more) illumination region 405 with illumination cores 404. The monolithic fiber may be made from polymer/s. In order to enhance the efficiency of energy transmission of the illumination through the fiber, the illumination region/s 405 may comprise hollow (air filled) illumination cores 404 bores in the monolithic fiber), extending from the proximal tip to the distal tip. PCF cores 406 may be provided, surrounding each illumination core 404 (e.g., several rings of PCF conduits, for example, in some embodiments, three or more rings). According to some embodiments, when there are several illumination channels (four in the illustrated example in FIG. 4), surrounded by PCF cores, there is no significant illumination loss in the fiber due to the PCF cores that serve to trap the illumination light in the illumination air filled cores. In some embodiments of the present invention, an illumination core diameter is about 100-200 microns, an imaging core diameter is about 1 micron, and a PCF core is sub wavelength (with respect to the traversing light), e.g., 0.2 micron.

In some embodiments, at the proximal end of the fiber, there may be a microscope magnification, so that the illumination region 405 and the imaging region 403 that collects reflected illumination and transmits it to the optical sensor (e.g., camera) can be spatially separated without any complicated alignment problems, despite the fact that both the illumination module as well as the camera are located at the same tip (the distal tip of the fiber). Y couplers may be used to split the light beam from the illumination source (222 in FIG. 2) into several (e.g., four) spatially separated illumination point sources.

FIG. 4B shows a cross section of a multicore fiber endoscope 450, with a plurality of metal coated cores 452, according to some embodiments of the invention. In order to overcome the resolution and the energetic efficiency of transmission problem that may be associated with cross talk of adjacent cores, each core 458 (e.g., imaging core, illumination core) may be coated. with a thin metallic coating 456 (e.g., thickness of some 150-300 nanometer, for example, 200 nm). Typically, to date, the pitch between cores in a multicore fiber is about 3.6 microns, and small cores have a diameter of about 0.9 microns. By providing the metallic coating, the density of the cores may be increased, resulting in more cores per cross area and thus higher imaging resolution. The pitch of adjacent cores, according to some embodiments of the present invention, may be smaller than 2 microns, for example around 1 micron. The coating acts like a mirror, preventing cross talk between the cores. The optical mode may be confined to travel only through the core and has no tails in the material of the cladding. This arrangement may reduce transmission losses as the material of the cladding typically absorbs more than the material of the core. The fabrication of such a multicore fiber may involve coating each rod with a metallic coating (e.g., a tube), and then building the pre-form as known in the art and then drawing it as known in the art. When stating “about”, for the purpose of the present invention, it is meant to cover a deviation of 10 percent from the actual measurement.

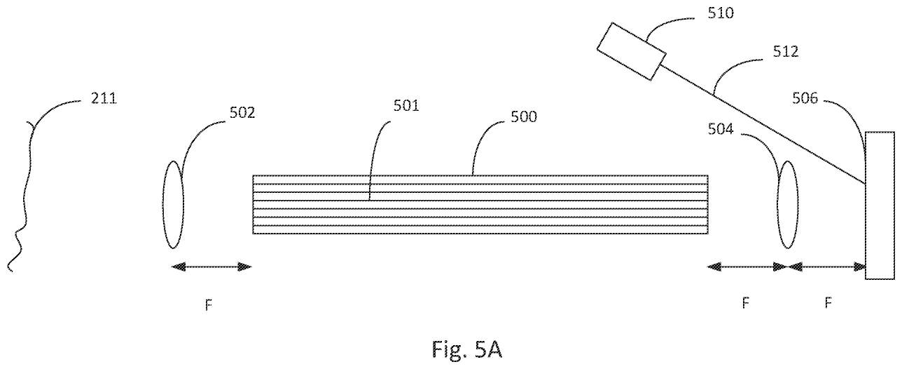

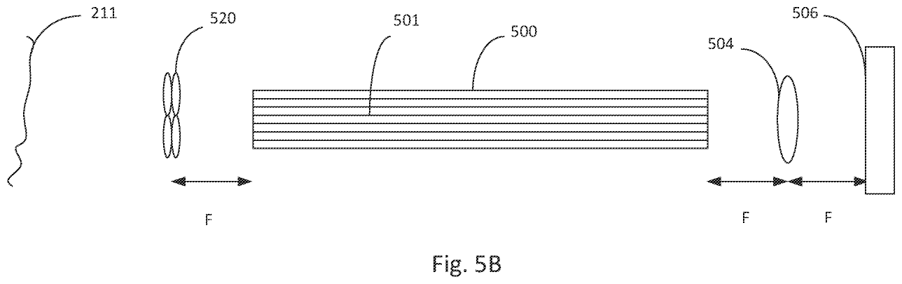

FIG. 5 shows a multicore fiber 500 for an endoscope, according to some embodiments of the invention, for obtaining high-resolution imaging.

At the distal tip of the fiber 500, which is designed to image inspected surface 211 within a patient's body cavity, a Fourier transformation lens 502, may be placed, at the focal distance F, while at the proximal tip of fiber 500 an inversed Fourier transformation lens 504 may be placed at distance F from the distal tip. Performing such transformation (Fourier transformation) is possible since the illumination light beam is generated by a coherent laser source. This arrangement causes the cores 501 of the multicore fiber 500 of the endoscope to perform sparse compressed sampling of the Fourier domain. Then, the imaging lens 504 near the optical detector 506 (e.g., camera) performs inverse Fourier, and thus a high-resolution image may be obtained without having to digitally enhance the image (e.g., interpolation between the cores image data obtained by the imaging cores) as the cores sample the Fourier plane and do not transmit the actual image. Such arrangement may facilitate an all optical interpolation realized via the sparse sampling, which itself is realized via the structure of the multicore fiber 500.

In some embodiments of the present invention, for example, if the fiber is not well polished, causing a random phase to be introduced, then the lens 502 at the distal tip of the fiber may be used for optical imaging. A laser source 510 may be used to illuminate the optical detector 506 with a reference illuminating beam 512 to realize digital Fourier holographic recording (as coherent laser illumination is used) that may facilitate the construction of the phase and of the high resolution object solely by digital decoding.

FIG. 513 shows a multicore fiber for an endoscope, according to some embodiments of the invention, for obtaining high-resolution imaging, using optical element 520 comprising a plurality of (e.g., four) PCFs (like in a kaleidoscope), which is placed on the optical path of the light reflected from the inspected surface into the multicore fiber.

Because of the use of optical element 520 that includes four PSFs, the inspected surface 211 that needs to be Fourier transformed becomes Hermitic symmetric which means that its Fourier transformation is real. That way the Fourier transformed inspected surface 211 can be transferred via the multicore fiber 500 regardless of any phase distortion as the detector 506 captures only intensity images.

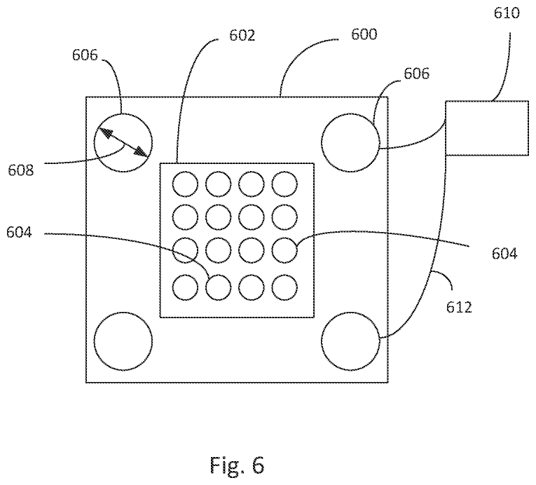

FIG. 6 shows a cross section of a rectangular multicore fiber 600 of an endoscope, with white light illumination cores, according to some embodiments of the present invention.

According to some embodiments of the invention, rectangular multicore fiber 600 includes imaging region, where a plurality of imaging cores 604 are located, for transferring light reflected off the inspected surface. An illumination arrangement, according to some embodiments of the invention, includes a set of lasers with different wavelengths which allow obtaining a simple realization of multispectral imaging (Narrow Band Imaging (NBI) etc.). The problem with this type of illumination with a set of discrete wavelengths is that, although a good gamut can be obtained, the holes in the spectrum can lead to bad detection quality for specific types of tissues. In order to solve this, hollow PCF cores 606 with a very high numerical aperture (NA) may be provided, for example in the periphery of the imaging region and filling it with a fluorescent dye (liquid). The dye may be contained in container 610, from which it may be delivered via delivery lines 612 (only some are shown in the figure) into cores 606 (e.g., having a predetermined diameter 608). The dye may be selected to be a solution mixture that can be configured to be irradiated and pumped with a discrete laser of the illumination module. The fluorescent dye along the long illumination PCF cores may generate continuous white light illumination spectrum that is an addition to the discrete wavelengths of the laser used for multi spectral imaging/sensing.

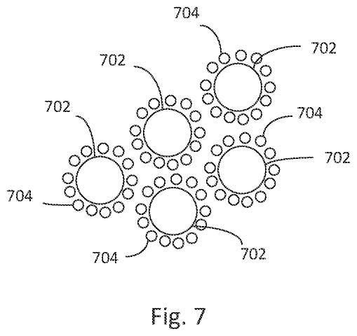

FIG. 7 shows cross section of several imaging cores with peripheral cores for inhibiting cross talk between the imaging cores that may be included in an endoscope with a multicore fiber, according to some embodiments of the invention.

One of the ways of enhancing imaging resolution of a multicore fiber, according to some embodiments, is to have higher density of cores 702 in the imaging region. However, it is desired to increase this density of the imaging cores without increasing cross talk between the cores. In order to inhibit possible cross talk, the difference of the refraction index between cores and their surrounding may be artificially increased by adding a plurality of surrounding cores 704 around each of the imaging cores. The surrounding cores may be hollow, air-filled. That way the average refraction index may be averaged between the refraction index of the cladding material and air (refraction index of 1). The diameter of the surrounding cores should preferably be made smaller than the optical wavelength of the anticipated light traversing through the imaging cores. The surrounding cores may be large when drilled in the pre-form but decreased in dimensions after the drawing to be smaller than the optical wavelength of the reflected light traversing through the imaging cores. That way, the imaging cores may be placed closer together, without increasing cross talk.

Since the transmission of polymer fibers is not very efficient, very strong illumination may be required in order to have sufficient amount of reflected light reach the optical detector. This may create radiation safety issues.

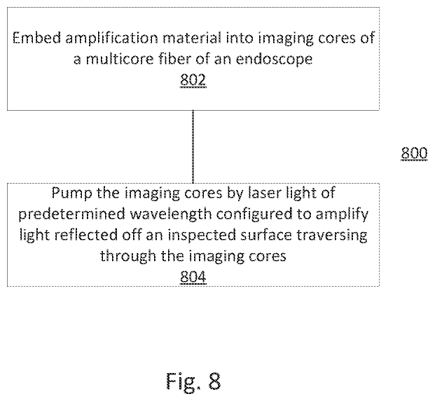

FIG. 8 illustrates a method 800 for increased radiation safety in endoscopy that uses laser light, according to some embodiments of the present invention. Method 800 may include embedding amplification material (e.g., Nd:YAG, doped Erbium, etc.) into imaging cores of a multicore fiber of an endoscope, and pumping the imaging cores by laser light of predetermined wavelength configured to amplify light reflected off an inspected surface traversing through the imaging cores. Since the collected light is amplified, fewer photons may be needed in the imaging fiber to offer enough sensitivity for a CMOS sensor. Less collected light menus illuminating the tissue with less light, which may impart safety conditions for the inspected tissue.

One of the advantages of the use of endoscopes with embedded amplification material is that this may allow the use of longer endoscopes for a large variety of medical applications.

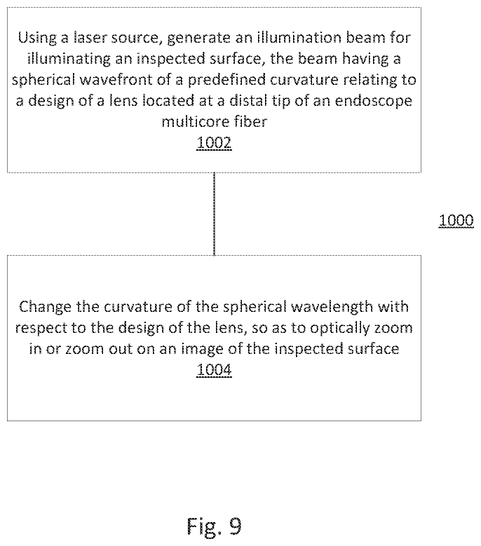

FIG. 9 shows a method 1000 for optical zooming in endoscopy, according to some embodiments of the invention.

Method 1000 may include using 1002 a laser source, generating an illumination beam for illuminating an inspected surface, the beam having a spherical wavefront of a predefined curvature relating to a design of a lens located at a distal tip of an endoscope multicore fiber. Method 1000 may further include changing 1004 the curvature of the spherical wavelength with respect to the design of the lens, so as to optically zoom in or zoom out on an image of the inspected surface.

To realize optical zooming by projecting with the laser illumination, a spherical wavefront may be provided that is matched to the lens design at the tip of the endoscope. Changing the curvature of the illumination beam changes the optical zoom factor.

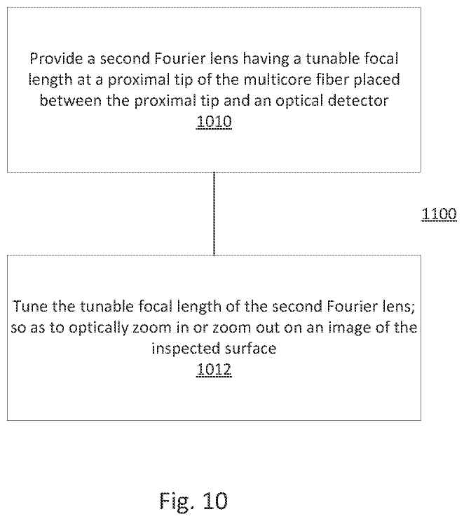

FIG. 10 shows a method 1100 for optical zooming in endoscopy, according to some other embodiments of the invention.

This method is suitable for an endoscope for inspecting an inspected surface that has a first Fourier lens having a focal length at a distal tip of a multicore fiber. Method 1100 may include providing 1010 a second Fourier lens having a tunable focal length at a proximal tip of the multicore fiber placed between the proximal tip and an optical detector, and tuning 1012 the tunable focal length of the second Fourier lens; so as to optically zoom in or zoom out on an image of the inspected surface.

The scaling (optical zooming) may be equal to the ratio between the focal length of the fixed lens at the distal tip of the endoscope and the tunable focal length at the proximal end.

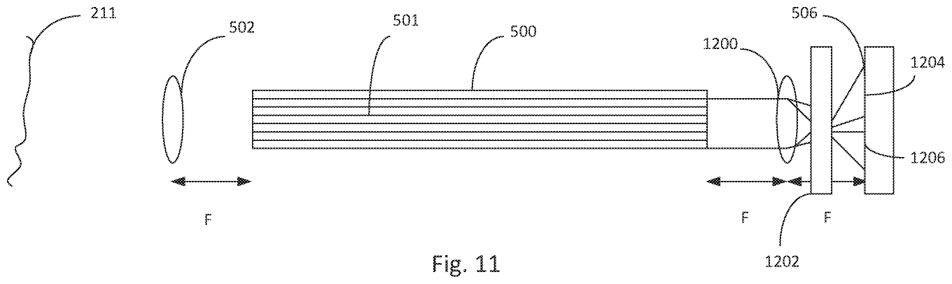

FIG. 11 shows a multicore fiber of an endoscope for providing two optical zoomed. images, according to some other embodiments of the invention.

In this embodiment, the lens 1200 at the proximal end of the fiber may have two focal lengths—one for lambda1 (first wavelength) and one for lambda2 (second wavelength), such that one of the focal lengths performs a Fourier transform and the other focal length performs optical imaging. The two wavelengths may be separated by a grating 1202 located in front of the detector 506. That way an optical image is projected on half 1204 of the detector, e.g., low resolution and good field of view, and a Fourier transform image is projected on another part of the detector 1206, e.g., high resolution and limited field of view. That way, an optical zooning by lambda multiplexing may be obtained.

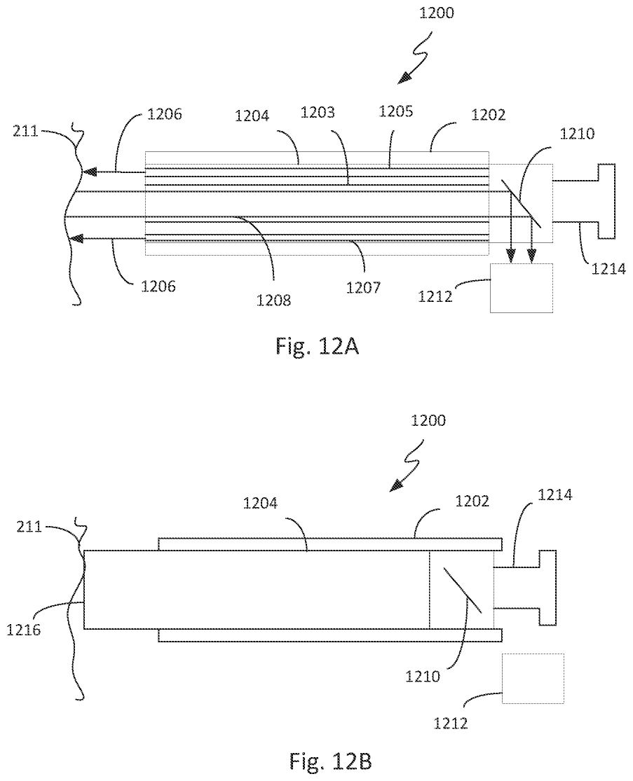

FIG. 12A shows a multicore fiber endoscope 1200 with sampling capability, according to some embodiments of the invention, in a viewing mode. The multicore, fiber endoscope 1200 includes a multicore fiber 1202 with a plurality of imaging cores and a plurality of illumination cores. In the viewing mode, the plurality of illumination cores (only two illumination core 1205, 1207 are shown, for brevity) are used to direct light 1206 onto the imaged surface 211. The plurality of imaging cores (only one imaging core 1203 is shown, for brevity) are used to direct light 1208 reflected off the imaged surface 211 to an imaging sensor 1212. A beam deflector 1210 (e.g., beam splitter) may be used to divert the reflect light beam 1208 to the imaging sensor 1212 (e.g., CMOS detector). The multicore fiber 1204 is inserted in a shielding sleeve 1202. A mechanism for advancing and retracting the multicore fiber in the shielding sleeve, such as plunger 1214, may be provided at the proximal end of the endoscope, coupled to beam deflector 1210 at the proximal end of the multicore fiber 1204.

12B shows a multicore fiber endoscope with sampling capability, according to some embodiments of the invention, in a sampling mode. The illumination cores and the imaging cores are not shown, for brevity. When facing a tissue surface 211 to be sampled, in the sampling mode, the distal tip 1216 of multicore fiber 1204 is advanced out of the shielding sleeve 1202, e.g., by a few millimeters, for example by pressing plunger 1214, so that tip 1216 may be placed in contact with the tissue surface 211. A sample for analysis, such as, for example, tissue cells, as well as liquid or other materials that may be found on the surface may thus adhere to the tip 1216 of the multicore fiber 1204. After engaging with the surface, the multicore fiber 1204 may be retracted into the shielding sleeve 1202, for example by pulling plunger 1214, so that the sample obtained remains intact on the tip 1216, when the endoscope is extracted out of the body of the patient.

Allowing to the imaging fiber tip to get out of its mechanical navigation shield a few millimeters and then to get in again will allow it to be inserted into liquids/tissue inside the body and a sample of those liquids/tissues to be removed and will allow a biopsy done to this tissue later on.

This biopsy related capability can also be realized in a different approach by using a spectrometer (e.g., a Raman spectrometer) replacing the imaging sensor, that when cooperating with the illuminating fiber laser can allow realization of a Raman spectroscopy suitable for performing a “non-contact biopsy” analysis of the sample.

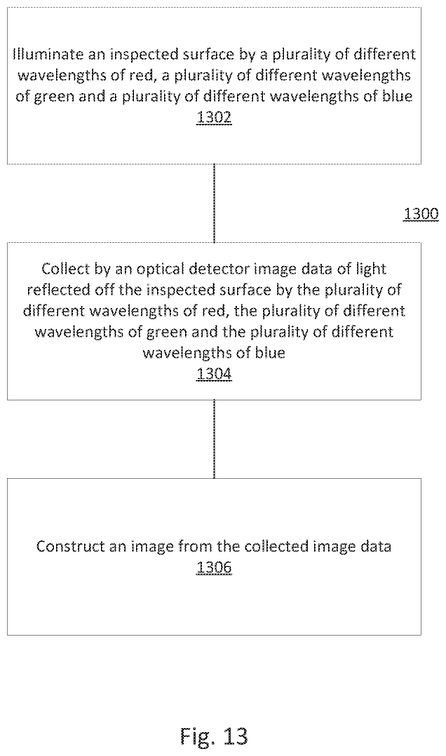

FIG. 13 illustrates a method for improving resolution of an image obtained in endoscopy, according to some embodiments of the invention.

Method 1300 may include illuminating 1302 an inspected surface by a plurality of different wavelengths of red, a plurality of different wavelengths of green and a plurality of different wavelengths of blue. Method 1300 may also include collecting 1304 by an optical detector image data of light reflected off the inspected surface by the plurality of different wavelengths of red, the plurality of different wavelengths of green and the plurality of different wavelengths of blue. Method 1300 may also include constructing 1306 an image from the collected image data.

Some embodiments of the invention may include using wavelength multiplexing, e.g. for sending several wavelengths of red, of green and of blue in order to improve resolution of imaging by collecting several wavelengths for each color per core and then the resolution in enhanced according to the number of launched wavelengths per color. The resolution enhancement is obtained if each one of the colors in the set (red, green or blue) goes to a different spatial location on the inspected object, thus generating a look-up table between colors and spatial position. That way, inspecting the spectroscopic information can give spatial information following the used mapping.

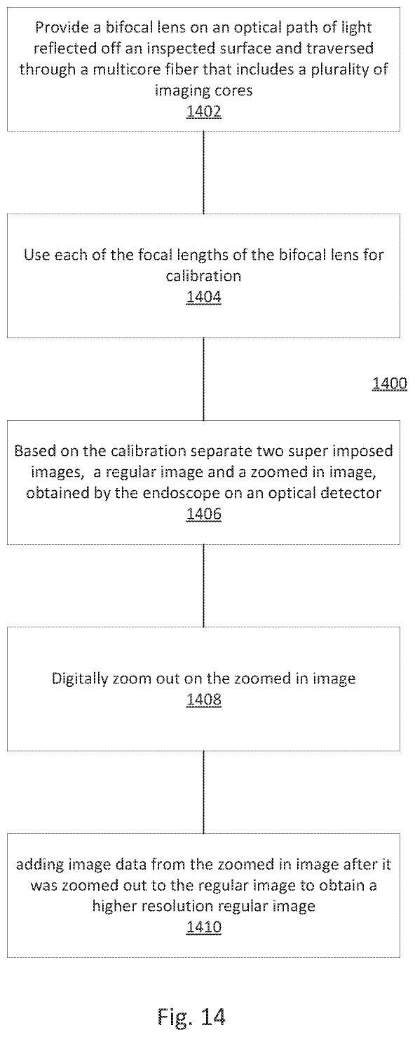

FIG. 14 shows a method for optical zooming and improving image resolution in endoscopy, according to some embodiments of the invention.

Method 1400 may include providing 1402 a bifocal lens on an optical path of light reflected off an inspected surface and traversed through a multicore fiber that includes a plurality of imaging cores.

Method 1400 may also include using 1404 each of the focal lengths of the bifocal lens for calibration, and, based on the calibration, separating 1406 two super imposed images, a regular image and a zoomed in image, obtained by the endoscope on an optical detector.

Method 1400 may also include digitally zooming out 1408 on the zoomed in image and adding 1410 image data from the zoomed in image after it was zoomed out to the regular image to obtain a higher resolution regular image.

Each focal length has some zeroed points in space to be used as calibration in order to separate the two super imposed images—the regular one and the zoomed one. After the separation (like in blind source separation) the zoomed image can be digitally zoomed out and added to the regular image to yield higher resolution in the central part of the field of view.

Some embodiments of the present invention may be embodied in the form of a system, a method or a computer program product. Similarly, some embodiments may be embodied as hardware, software or a combination of both. Some embodiments may be embodied as a computer program product saved on one or more non-transitory computer readable medium (or media) in the form of computer readable program code embodied thereon. Such non-transitory computer readable medium may include instructions that when executed cause a processor to execute method steps in accordance with examples. In some examples, the instructions stored on the computer readable medium may be in the form of an installed application and in the form of an installation package.

Such instructions may be, for example, loaded by one or more processors and get executed.

For example, the computer readable medium may be a non-transitory computer readable storage medium. A non-transitory computer readable storage medium may be, for example, an electronic, optical, magnetic, electromagnetic, infrared, or semiconductor system, apparatus, or device, or any combination thereof.

Computer program code may be written in any suitable programming language. The program code may execute on a single computer system, or on a plurality of computer systems.

Some embodiments are described hereinabove with reference to flowcharts and/or block diagrams depicting methods, systems and computer program products according to various embodiments.

Features of various embodiments discussed herein Wray be used with other embodiments discussed herein. The foregoing description of the embodiments has been presented for the purposes of illustration and description. It is not intended to be exhaustive or limiting to the precise form disclosed. It should he appreciated by persons skilled in the art that many modifications, variations, substitutions, changes, and equivalents are possible in light of the above teaching. It is, therefore, to be understood that the appended claims are intended to cover all such modifications and changes that fall within the true spirit of the present invention.

1. An endoscope comprising:
an illumination source for generating a coherent laser illumination beam;
an optical sensor;
a multicore fiber comprising:
at least one core for transferring the illumination beam from the illumination source through said at least one core to a distal end of the fiber, for illumination of a surface to be inspected; and
a plurality of cores for transferring light reflected off the surface to the optical sensor:
a temporal modulation sequencer for separating a specular image of the illumination beam from an image of the surface; and
a processor, for processing sensed data from the optical sensor to generate the image of the surface.

2. The endoscope of claim 1, wherein the temporal modulation sequencer comprises two Faraday rotators, each having a rotation axis, wherein the rotation axes are substantially parallel but offset.

3. The endoscope of claim 1, wherein the two Faraday rotators are configured to produce a lock-in amplification effect.

4. An endoscope comprising:
a multicore fiber comprising:
an illumination region that includes at least one hollow core for transferring an illumination beam from an illumination source through said at least one core to a distal end of the fiber, for illumination of a surface to be inspected, and a plurality of cores surrounding each of said at least one hollow core for trapping the illumination beam in said at least one hollow core; and
an imaging region that includes a plurality of cores for transferring light reflected off the surface to the optical sensor.

5. The endoscope of claim 4, wherein the plurality of cores surrounding each of said at least one hollow core are arranged in a plurality of rings around that hollow core.

6. The endoscope of claim 5, wherein the plurality of rings comprises at east three rings.

7. The endoscope of claim 4, wherein the plurality of cores surrounding each of said at least one hollow core comprise photonic crystal fibers (PCF).

8. An endoscope comprising:
an optical sensor;
a multicore fiber comprising:
a plurality of cores for transferring light reflected off an inspected surface to the optical sensor;
a lens for performing Fourier transform on the light reflected off the surface placed at a predetermined distance from a distal tip of the multicore fiber;
a lens for performing inverse Fourier transform on the light reflected off the surface after it emerges from a proximal tip of the multicore fiber, placed at a predetermined distance from a proximal tip of the multicore fiber;
a processor, for processing sensed data from the optical sensor to generate the image of the surface.

9. The endoscope of claim 8, further comprising a laser source for generating a reference illuminating beam to realize a digital Fourier holographic recording so as to construct a phase and of an image of the inspected surface by digital decoding.

10. The endoscope of claim 8, further comprising an optical element that includes a plurality of PCF cores, placed on the optical path of the light reflected off the inspected surface into the multicore fiber.

11. An endoscope comprising:
a multicore fiber comprising:
an illumination region that includes at least one imaging core for transferring an illumination beam from an illumination source through said at least one core to a distal end of the fiber, for illumination of a surface to be inspected
an imaging region that includes a plurality of cores for transferring light reflected off the surface to the optical sensor; and
a dye container for containing a fluorescent dye configured to supply the dye into said one imaging core, so as to allow irradiating the dye to generate continuous white light illumination spectrum.

12. The endoscope of claim 11, the dye is a solution mixture.

13. An endoscope comprising:
a multicore fiber comprising:
an imaging region that includes a plurality of imaging cores for transferring light reflected off a surface to an optical sensor;
a plurality of surrounding cores about each of said plurality of imaging cores, wherein a diameter of each of the surrounding cores is smaller than the wavelength of reflected light anticipated to traverse through the imaging cores.

14. The endoscope of claim 13, wherein the surrounding cores are hollo air-filled cores.

15. A method for increased radiation safety in endoscopy that uses laser light, the method comprising:
embedding amplification material into imaging cores of a multicore fiber of an endoscope; and
pumping the imaging cores by laser light of predetermined wavelength configured to amplify light reflected off an inspected surface traversing through the imaging cores.

16. A method for optical zooming in endoscopy, the method comprising:
using a laser source, generating an illumination beam for illuminating an inspected surface, the beam having a spherical wavefront of a predefined curvature relating to a design of a lens located at a distal tip of an endoscope multicore fiber; and
changing the curvature of the spherical wavelength with respect to the design of the lens, so as to optically zoom in or zoom out on an image of the inspected surface.

17. A method for optical zooming when using an endoscope for inspecting an inspected surface, the endoscope having a first Fourier lens having a focal length at a distal tip of a multicore fiber, the method comprising:
providing a second Fourier lens having a tunable focal length at a proximal tip of the multicore fiber placed between the proximal tip and an optical detector; and
tuning the tunable focal length of the second Fourier lens; so as to optically zoom in or zoom out on an image of the inspected surface.

18. A method for improving resolution of an image obtained using an endoscope with a multicore fiber, the method comprising:
illuminating an inspected surface by a plurality of different wavelengths of red, a plurality of different wavelengths of green and a plurality of different wavelengths of blue;
collecting by an optical detector image data of light reflected off the inspected surface by the plurality of different wavelengths of red, the plurality of different wavelengths of green and the plurality of different wavelengths of blue, and
constructing an image from the collected image data.

19. A method for optical zooming and improving image resolution in endoscopy, the method comprising:
providing a bifocal lens on an optical path of light reflected off an inspected surface and traversed through a multicore fiber that includes a plurality of imaging cores;
using each of the focal lengths of the bifocal lens for calibration;
based on the calibration separating two super imposed images, a regular image and a zoomed in image, obtained by the endoscope on an optical detector;
digitally zooming out on the zoomed in image; and
adding image data from the zoomed in image after it was zoomed out to the regular image to obtain a higher resolution regular image.

20. An endoscope comprising:
a multicore fiber comprising:
a plurality of imaging cores for transferring light reflected off a surface to an optical sensor, each core coated by a metallic coating.

21. The endoscope of claim 20, wherein a pitch of the cores is smaller than 2 microns.

22. The endoscope of claim 21, wherein the pitch is about 1 micron.

23. An endoscope comprising
a multicore fiber with a distal end and a proximal end, having one or a plurality of illumination cores for transmitting light from the proximal end and out of the distal end and illuminating an internal surface of a patient to be viewed and with one or a plurality of imaging cores to transfer light reflected off the surface into the imaging cores from the distal end to the proximal end;
a shielding sleeve in which the multicore fiber is inserted; and
a mechanism for advancing and retracting the multicore fiber within the shielding sleeve, so as to allow extracting a distal tip of the multicore fiber to contact the surface and collect a sample for analysis and retract the distal tip into the shielding sleeve.

24. The endoscope of claim 23, wherein the mechanism comprises a plunger.

25. The endoscope of claim 23, comprising an optical sensor for receiving the reflected light.

26. The optical sensor of claim 25, wherein the optical sensor is a Raman spectrometer, for performing analysis of the sample.
